(19) United States
(12) Patent Application Publication
(10) Pub. No.: US 2003/0028020 A1
(43) Pub. Date: Feb. 6, 2003

(54) Process for the synthesis of high purity melamine

(76) Inventors: Ram B. Gupta, Stamford, CT (US); Venkatrao K. Pai, Stamford, CT (US); John Bradley Roucis, Chino Hills, CA (US); Alice Elizabeth Hicks, London (GB)

Correspondence Address: PENNIE & EDMONDS LLP, 1667 K Street, N.W., Washington, DC, 20006, US

(21) Appl. No.: 10/144,026

(22) Filed: May 14, 2002

Related U.S. Application Data
(63) Continuation-in-part of application No. 09/915,587, filed on Jul. 27, 2001, now abandoned.

(51) Int. Cl. C07D 251/62

(52) U.S. Cl. 544/203

(57) ABSTRACT

The present invention relates to the production and purification of melamine and, in particular, to the purification of a crude melamine product, while minimizing impurities comprising at least one of melem, melam, melon, or ureidomelamine. The crude melamine can be treated with ammonia and a promoter to achieve a very high melamine content in the purified product, at more advantageous conditions, than treatment with ammonia alone. The present invention is also directed to an improved melamine production process that yields melamine with so few impurities that it can be pure enough as to not require an additional purification step.

### FIELD OF THE INVENTION

The present invention relates to the production and purification of melamine and, in particular, to the purification of a crude melamine product, while minimizing impurities comprising at least one of melem, melam, melon, or ureidomelamine. The crude melamine can be treated with ammonia and a promoter to achieve a very low impurity content in the purified melamine product, at more advantageous conditions, than treatment with ammonia alone. The present invention is also directed to an improved melamine production process that yields melamine with so few impurities that it can be pure enough as to not require an additional purification step.

### BACKGROUND OF THE INVENTION

The production of melamine is industrially important, as melamine is extensively used in the fields of coatings, pharmaceuticals, building materials, paper-making, leather, and textiles. Melamine-based resins are in highest demand as high-grade finish coatings, particularly in the automobile industry, although they are also widely used due to their outstanding properties of flame retardance, adhesion, water and oil resistance, and durability.

Recent global demand for melamine and melamine-based resins has rivaled the current industrial supply capabilities. As a result, there has been a large corporate effort over the past few years to increase the output and efficiency of existing production and processing methods. Commercial melamine synthesis processes have been streamlined to provide crude melamine with a lower level of undesired reaction products, and certain purification methods have been shown to help convert the crude melamine to a higher purity. Since the impurities in the crude melamine can compromise the properties and performance of melamine or melamine-based resins in desired applications, it is generally advantageous to minimize the amount of impurities in the melamine product.

A number of recent patents claim various synthesis conditions and reactor designs for both high-pressure and low-pressure processes to produce crude melamine. Generally, high-pressure melamine production processes involve pressures greater than about 720 psig, typically without a catalyst, while low pressure melamine production processes involve pressures between about 0 psig and 150 psig, typically in the presence of a catalyst, both types of processes involving temperatures from about 350° C. to 450° C.

High-pressure processes typically occur in a liquid phase, in a smaller reactor, and require a lower capital investment. In addition, the off-gases of melamine synthesis reactions including urea as a starting material, generally also including ammonia and carbon dioxide, are already at a high pressure and are useful as starting materials for a urea synthesis process. The disadvantages of high-pressure processes tend to include increased likelihood of corrosion due to higher pressures and increased levels of impurities in the product due to the liquid phase reaction.

Low-pressure processes typically also utilize urea as a starting material, and the reaction generally occurs in a vapor phase, resulting in lower levels of impurities being present in the product. Due to the lower pressures, such processes tend to be less corrosive than higher pressure processes. The disadvantages of low-pressure melamine synthesis processes include a necessity for larger volume reactors, which require more investment capital, and problems associated with catalyst beds, such as local hot spots, agglomeration, and/or loss of catalyst as an impurity in the melamine product. Additionally, in order for any off-gases from the reaction to be recycled, it is necessary that the off-gases be further compressed.

Whatever synthesis process is used, crude melamine is typically purified by one or more of a variety of different methods immediately after its synthesis, as part of the production process, or by a post-processing purification method. Purification of the crude melamine is rather important commercially, as the industries and products which make use of melamine generally require the melamine to be of high purity, although there are instances where crude, or impure, melamine may be useful, for example, as described in U.S. Pat. No. 5,120,821.

One of the earliest publications in the field of high-pressure melamine synthesis and purification is a 1949 American Cyanamid patent, U.S. Pat. No. 2,475,709, which discloses a process of treating melam, melem, and melon, all of which are adducts of melamine formed through loss of ammonia, at least one of which is typically present as an impurity in crude melamine. The melam, melem, melon, or a mixture thereof, alone or as impurities in crude melamine, are exposed to anhydrous ammonia at a temperature above 350° C. and at a high pressure, i.e., at least about 1000 psi, at which conditions they tend to decompose in substantially quantitative yields to form melamine. The following disclosures relating to high-pressure melamine synthesis and purification processes utilize substantially the same chemistry as the 1949 American Cyanamid patent.

U.S. Pat. No. 4,565,867 discloses a high-pressure anhydrous melamine synthesis process that provides crude liquid melamine from molten urea, which is supplied at temperatures from 700° F. to 800° F. (371° C. to 427° C.) and at pressures from 1700 psig to 2200 psig. Carbon dioxide and ammonia off-gases are also formed during the reaction process. The crude melamine is then purified by quenching with a liquid, preferably liquid ammonia, at lower temperature (about 120° F. to 260° F., or about 49° C. to 127° C.) and pressure (less than about 615 psig) to obtain a purified melamine powder. The purity of the melamine made by this process is in the range of 96 to 99.5% without the need for further purification.

European Patent No. 0808836 and International Publication No. WO 97/34879 each disclose a continuous melamine production process that involves pyrolyzing urea at pressures of about 5 to 25 MPa (about 725 psi to 3625 psi) and at temperatures of about 325° C. to 450° C. to form crude liquid melamine and carbon dioxide and ammonia off-gases. The process also involves further purifying the crude liquid melamine and any melamine vapor in the off-gases by further reaction of the liquid with ammonia and by simultaneously cooling and isolating a purified product using a supercritical fluid. After purification, the melamine purity is reported to be between 99.5% and 99.95%. Other related publications utilize similar processes for synthesizing the crude melamine, but the processes differ in the purification step(s) and/or reaction conditions. Among these are European Patent No. 0929531 and International Publication Nos. WO 99/19310, WO 98/55466, WO 98/55465, WO 98/52928, WO 98/54160, WO 98/27071, WO 98/04533, and WO 97/47609.

U.S. Pat. No. 5,731,437 discloses a melamine preparation process involving pressurizing molten urea with ammonia at 50 bar to 150 bar (725 psi to 2175 psi) and at 360° C. to 430° C. As with other high-pressure processes, the off-gases are separated, and the crude liquid melamine is vaporized with ammonia and cooled, usually at lower temperature (below 130° C.) and pressure (less than 40 bar, or 580 psi), to form a crystalline product.

International Publication No. WO 99/00374 discloses a continuous high pressure melamine manufacturing process wherein urea is fed into molten melamine at a pressure greater than 7 MPa (1015 psi) and at a temperature of 360° C. to 420° C., and a gas phase of mostly carbon dioxide and ammonia is removed. A portion of the liquid phase, containing 85% to 95% melamine, is further purified with fresh ammonia in a plug flow reactor at a pressure greater than 7 MPa (1015 psi) and at a temperature of 360° C. to 450° C.

U.S. Pat. No. 5,514,797 discloses a purification process in which the crude melamine is heated to about 250° F. to 1000° F. under a pressure from about 600 psi to 3000 psi in the presence of ammonia. This process provides melamine having a purity of at least 99%.

U.S. Pat. No. 5,721,363 discloses a process for the production of highly pure melamine comprising an aftertreatment purification of crude or molten melamine that involves slow, controlled cooling of the melamine under partial pressure of ammonia. This patent also discloses various critical temperatures, cooling rates and profiles, ammonia pressures, residence times, and other variables that may be varied in combination to effect a melamine purity above 99.8% and a melem content below 100 ppm.

Therefore, it would be desirable to obtain the benefit of improving the purity of crude or low purity melamine through a purification process that can make use of existing melamine synthesis equipment, and that does not involve a significant additional investment in equipment or resources. Furthermore, it would also be desirable to obtain the benefit of efficiently attaining high purity melamine, preferably without expensive or complicated post-production purification steps.

### SUMMARY OF THE INVENTION

The present invention is directed primarily to a process for the production of high purity melamine, or to a crude melamine purification process to remove undesired components, e.g., undesired reaction products associated with a melamine synthesis process, both processes including: heating melamine having a first purity and undesired components therein in a reaction zone to a temperature sufficient to maintain at least a portion of the melamine in a molten state; adding ammonia to the reaction zone at a temperature and pressure sufficient to decompose undesired components to purify the melamine; adding a base promoter to the reaction zone in an amount sufficient to facilitate the decomposition of the undesired components in the melamine without significantly decomposing melamine; and recovering purified melamine having a second purity higher than the first purity. In one embodiment, the undesired components in the melamine include at least one compound that is decomposable into melamine in the presence of, preferably facilitated or catalyzed by, the base promoter. In the purification process, the melamine is preferably crude melamine or low purity melamine, although high purity melamine may be further purified according to the invention.

Preferably, the base promoter is at least partially soluble in ammonia, urea, the melamine having a first purity, or a combination thereof, especially at the decomposition reaction temperature and pressure. Additionally, the base promoter can include a nitrogen-containing Lewis base; an amide salt having a general formula [M]^{+x}([NH₂]⁻)ₓ, where M is a monovalent or multivalent metal and where x is from 1 to 6; or a combination thereof. Preferably, the base promoter may include sodium amide, potassium amide, lithium amide, magnesium amide, calcium amide, triethylamine, tributylamine, N,N-diisopropylethylamine, N,N-diethylcyclohexylamine, triethylenediamine, pyridine, quinoline, lutidine, N,N,N′,N′-tetramethylethylenediamine, dimethylaminopyridine, or combinations thereof.

Ammonia may be added, typically at a pressure from about 200 psi to 2000 psi. Also, the recovery step preferably includes retrieval of a solid, with solidification of the melamine preferably occurring by sufficiently reducing the pressure of the reaction zone.

In another preferred embodiment, the decomposition reaction of the undesired components in the melamine results in conversion of at least about 10%, preferably at least about 50%, more preferably at least about 90%, of the undesired components into melamine. Optionally, the process can further include purging the reactor with nitrogen at one or more intervals throughout the purification process, optionally at a pressure from about 100 psig to 1000 psig.

The present invention is also directed to a process for the production of melamine including the steps of: adding a base promoter to one or more reactants for synthesizing melamine to form a mixture, wherein the base promoter is present in an amount sufficient to facilitate a reaction between the one or more reactants to form melamine; and maintaining the mixture at a temperature and a pressure sufficient to facilitate the reaction between the one or more reactants such that the melamine is formed. Advantageously, the one or more reactants is urea or comprises at least one impurity obtainable from a melamine synthesis reaction or purification process. In a preferred embodiment, the base promoter can be at least partially soluble in, or compatible with, at least one of the reactants. In addition, the base promoter may include a nitrogen-containing Lewis base; an amide salt having a general formula [M]^{+x}([NH₂]⁻)ₓ, where M is a monovalent or multivalent metal and where x is from about 1 to 6, preferably from about 1 to 3; or a combination thereof.

In a preferred embodiment, the melamine product is substantially free of undesired reaction products. In another preferred embodiment, the melamine product has a high purity, preferably a purity of at least about 98%, more preferably at least about 99%, most preferably at least about 99.8%. In another preferred embodiment, the melamine product includes less than about 20,000 ppm of undesired components, i.e., undesired reaction products.

In a preferred embodiment, the melamine synthesized by any of the processes according to the invention is substantially free of undesired reaction products such that the melamine contains at least one of: less than about 1200 ppm melam impurities; less than about 200 ppm melem impurities; less than about 200 ppm melon impurities; less than about 400 ppm ureidomelamine impurities; less than about 700 ppm combined ammeline, ammelide, and cyanuric acid impurities; less than about 150 ppm melamine cyanurate impurities; or a combination thereof.

### DETAILED DESCRIPTION OF THE PREFERRED EMBODIMENTS

One aspect of the present invention involves a process of purification of commercially synthesized crude melamine to remove certain undesired components, e.g., undesired reaction products associated with the synthesis process, for example, such as melam, melem, melon, and/or ureidomelamine. Optionally, the undesired reaction products may also or alternatively include ammeline, ammelide, cyanuric acid, and/or melamine cyanurate.

The purification process, optionally performed in discrete batches, can include the following steps. The melamine, preferably crude melamine, is heated in a reaction zone, such as in a vessel or reactor, to a temperature sufficient to maintain at least a portion of the crude melamine in a molten state. Preferably, the reaction zone is heated to a temperature from about 345° C. to 450° C., more preferably from about 350° C. to 400° C. Ammonia is added to the reaction zone at a temperature and pressure sufficient to decompose undesired components to purify the crude melamine. Preferably, the pressure is from about 200 psi to 2000 psi, more preferably at a pressure from about 750 psi to 1250 psi. A base promoter is added to the reaction zone in an amount sufficient to facilitate the decomposition of the undesired components in the melamine without significantly decomposing melamine. The term “promoter,” as used herein, shall be understood to refer to any substance that accelerates or facilitates the desired chemical reaction and that is neither substantially consumed nor substantially generated during the chemical reaction. Preferably, the base promoter is present in an amount from about 0.01% to 10%, more preferably from about 0.1% to 1% by weight of the crude melamine mixture. The purified melamine can then be recovered. Preferably, the melamine can be recovered in its solid form. The purified melamine can preferably be solidified by controlled cooling, such that the cooling rate is between about 2° C./min and 5° C./min, preferably around about 3° C./min. Alternately or simultaneously, the melamine can be solidified by sufficiently reducing the pressure of the mixture.

The crude melamine suitable for use in the purification process according to the present invention may be synthesized by any commercial process available to one of ordinary skill in the art that provides melamine and reaction products that are decomposable by the purification process according to the present invention. For example, the melamine synthesis and/or purification processes may include any of those disclosed in U.S. Pat. Nos. 5,731,437; 5,721,363; 5,514,797; 5,120,821; 4,565,867; and 3,498,982, in European Pat. Nos. 0929531 and 0808836, and in International Publication Nos. WO 99/19310, WO 99/00374, WO 98/55466, WO 98/55465, WO 98/52928, WO 98/54160, WO 98/32731, WO 98/27071, WO 98/08808, WO 98/04533, WO 97/47609, and WO 97/34879.

Once the mixture of crude melamine and base promoter is heated to the appropriate temperature and the ammonia is added at the appropriate pressure, a reaction to decompose the undesired components, i.e., the undesired reaction products in the crude melamine, begins such that a minimum temperature, i.e., a temperature sufficient to allow the crude melamine to melt, is maintained for a period sufficient to convert a portion of the undesired reaction products in the crude melamine mixture to melamine, preferably the portion being at least about 10%, more preferably at least about 50%, most preferably at least about 90% of the undesired reaction products in the crude melamine.

Another aspect of the present invention relates to a process for the production of melamine. The process can include the following steps: adding a base promoter to one or more reactants for synthesizing melamine to form a mixture; and maintaining the mixture at a temperature and a pressure sufficient to facilitate the reaction between the one or more reactants such that the melamine is formed. Preferably, the base promoter is present in an amount sufficient to facilitate a reaction between the one or more reactants to form melamine. Preferably, the one or more reactants is urea or comprises at least one impurity obtainable from a melamine synthesis reaction or purification process. Advantageously, the melamine product may be substantially free of undesired reaction products. Alternately, if desired, the melamine product may be crude, or low purity, melamine. In a preferred embodiment, this process is a high-pressure process. In another preferred embodiment, the melamine product made by such a process may exhibit properties and impurity levels similar to those of other purified melamine products, for example, such as those of the present invention listed herein, i.e., the melamine may be high purity. Although the preferred process is a continuous one, the process may also be performed in discrete batches.

In a preferred embodiment, a continuous, high-pressure, anhydrous process is offered for converting urea to liquid melamine and by-product offgas. This offgas typically includes carbon dioxide and ammonia. The components of a plant system for use in carrying out this continuous process include, but are not limited to, an offgas scrubber unit, a reactor unit, a separator unit, and a product cooling unit. The continuous process of the invention includes the following steps, which may occur individually in any order or any two or more of which may occur concurrently:

(1) The continuous process of the invention involves introducing a base promoter into the process at any time such that it helps facilitate the reaction to form melamine and/or the conversion of melamine impurities, such as melam, melem, melon, ureidomelamine, and the like, to melamine. The base promoter, which may advantageously include any listed above, is maintained in an amount from about 0.01% to 10%, preferably from about 0.1% to 1%, based on the total weight of the melamine produced in the reactor at the time of introduction. The base promoter may be added at any point in the process of the invention such that facilitation of the reaction or conversion to form melamine is achieved. Preferably, the base promoter is preferably dispersed or dissolved in the molten urea in step (3), before the molten urea is fed to the reactor, or the base promoter may alternately be fed continuously into the reactor.

(2) Urea melt is fed into the scrubber at from about 1250 psig to 2500 psig pressure, preferably from about 1350 psig to 2200 psig, and at a temperature above the melting point of urea. In the scrubber, the liquid urea comes into contact with reaction offgases principally composed of CO₂ and NH₃ and typically including a small quantity of melamine. The molten urea can help to scrub the melamine from the offgas. In the scrubbing process, the offgases are cooled from about the temperature of the reactor, i.e., from about 350° C. to 430° C. to from about 175° C. to 235° C., and the urea is preheated to about 175° C. to 235° C. temperature range. The temperature and pressure in the scrubber can be interrelated. If the pressure is at the low end of the range, i.e., about 1250 psig to 1700 psig, the minimum temperature of the scrubber should vary from about 175° C. to 182° C.; whereas if the scrubber is at the high end of the pressure range, i.e., about 2000 psig to 2200 psig, the minimum temperature can be increased to about 182° C. to 195° C. Below the aforementioned minimum temperatures, ammonia and CO₂ can tend to condense in the bottom of the scrubber and may form undesirable carbamate, which may be difficult to remove. Generally, the higher the pressure, the higher the required minimum temperature. Above about 260° C., the urea may react to form intermediate products, which in some cases can be undesirable.

The carbon dioxide and ammonia offgases are removed from the scrubber and can preferably be recycled to a urea plant for conversion into urea. The preheated urea is taken from the scrubber, typically together with any melamine therein, and fed to the reactor at a pressure from about 1250 psig to 2500 psig. The scrubber, in one embodiment, is jacketed to provide supplemental cooling in the scrubber for temperature control. It may alternately be desirable to control the temperature of the scrubber by some other heat transfer means, such as coils or the like, or any known means available to one of ordinary skill in the art.

Accordingly, the scrubber can facilitate the removal of water, which may be present in the molten urea feed; the preheating of the molten urea with offgas; the removing of melamine from the offgases, if desired, to provide substantially melamine-free CO₂ and NH₃, preferably for recycling to a urea plant; and the recovering of excess heat energy.

(3) The urea (and optionally melamine) taken from the scrubber is fed to the reactor, optionally with a high-pressure pump. Before entering the reactor, however, a small quantity of ammonia may be injected as a liquid or preferably a hot vapor into the line to act as a purge to prevent plugging of the reactor and/or feed lines. The high-pressure pump can be eliminated, for example, by elevating the scrubber above the reactor.

(4) In the reactor, the molten urea is heated to a temperature from about 350° C. to 430° C., preferably from about 370° C. to 430° C., at a pressure of from about 1250 psig to 2500 psig, preferably from about 1350 psig to 2200 psig, under which conditions the melamine is formed. The reactor can be any of the state of the art high-pressure reactor, for example, such as disclosed in U.S. Pat. No. 3,470,163. The reactor typically operates full of liquid melamine, with the products from the reactor, which generally include melamine, ammonia, and carbon dioxide, being fed, preferably continuously, as a mixed stream to the gas separator.

(5) In the gas separator, melamine can be separated from the offgas, and collected, e.g., in the bottom of the separator. The separator is generally held at a temperature and pressure such that the melamine becomes or remains molten, and preferably at the same temperature and pressure as the reactor. The gaseous ammonia and carbon dioxide, which are typically saturated with melamine vapor, are removed and preferably recycled into the urea scrubber. The temperature and pressure are controlled in order that the melamine concentration in the scrubber is typically not more than about 10% melamine. Normally, the lower the operating pressures, the greater the amount of melamine removed with the offgases. The separated melamine can then be injected into the product cooling unit.

(6) In the product cooling unit, the melamine in liquid form is depressurized and rapidly cooled, optionally in the presence of a liquid medium. Using a liquid medium that is a vapor at the temperature of the product as a quench, dry melamine powder may be formed, typically without substantial formation of impurities. The melamine product may then be removed from the bottom of the cooling unit.

The product cooling unit preferably is maintained at a temperature below the melting point of urea to avoid stickiness of the isolated melamine. This way, if there are urea impurities in the melamine, the urea can be pulled off with the gas, e.g., ammonia, formed when the relatively hot liquid melamine contacts the relatively cool liquid quenching agent. The minimum temperature preferably is the vapor temperature equilibrium of the liquid quenching agent at the pressure of operation. The liquid quenching agent is a low boiling liquid, preferably ammonia, which boils with the gas being readily separated from the melamine product. The pressure can be atmospheric pressure or a pressure up to about 600 psig. It is preferred to operate at a pressure of about 200 psig to 400 psig and a temperature of from about 45° C. to 75° C.

In the presently disclosed process the pressure, as above defined, will typically be the same in the scrubber, reactor, and gas separator. The temperature of the reactor and the gas separator will generally also be the same. The offgases removed from the gas separator are typically at the same temperature as the reactor and separator until they reach the scrubber where they are cooled in the process of being scrubbed with the molten urea. The liquid melamine transferred from the gas separator generally enters the product cooling unit at the same temperature as the reactor and gas separator.

In the presently disclosed process it is preferred that the liquid melamine and offgas from the reactor are transferred from the reactor to the gas separator as a mixed stream, and the offgases and melamine separated in the separator unit. Optionally, a liquid medium may be used to quench the liquid melamine.

The dry melamine powder recovered directly from the quenching of the liquid melamine in the cooling unit is substantially pure melamine, having a purity of at least about 98% melamine or above and, accordingly, can be used directly in most melamine applications without purification. The purity of the recovered melamine, particularly the low levels of melem and melam which comprise no more than about 0.5% to 1.5% melem and melam, is surprising.

In addition, this process of the invention is surprisingly simple in contrast to the complex, high-energy consuming processes of most other commercial systems.

When the process involves discrete batches of high purity melamine being formed, the pressure and temperature are typically sufficient to facilitate the reaction. It is preferred that the pressure of the reaction zone is from about 200 psi to 2000 psi, more preferably from about 750 psi to 1250 psi, most preferably from about 900 psi to 1000 psi. In such a process, it is also preferred that the temperature of the reactants be from about 345° C. to 450° C., more preferably from about 350° C. to 425° C., most preferably from about 375° C. to 410° C. Also in such a process, it is preferred that the temperature and/or pressure of the melamine synthesis reaction zone is maintained for at least 30 minutes, more preferably for at least 45 minutes. Typically, the temperature and/or pressure of the melamine synthesis reaction zone is maintained for up to 90 minutes, but may optionally be maintained for longer. Additionally, it is preferred that the base promoter be dissolved in one or more of the reactants, for example, such as ammonia or urea, more preferably before the reactants are mixed together to create the second mixture. Alternately, the base promoter may be added in a separate step and/or after the reactants are provided to form the first mixture. Preferably, the base promoter is present in an amount from about 0.01% to 10%, more preferably from about 0.1% to 1% by weight of the second mixture.

Advantageously in any of the processes of the present invention, the base promoter can include any compound having the ability to facilitate the reaction to decompose at least a portion of the undesired reaction products in the melamine, preferably to decompose those reaction products which are decomposable into melamine. Compounds capable of facilitating the reaction include, for example, amide salts, Lewis bases, and mixtures thereof. Suitable amide salt base promoters include compounds having the formula [M]^{+x}([NH₂]⁻)ₓ, wherein M can be a monovalent or multivalent metal and x can be from 1 to 6, preferably from 1 to 3, such that the compounds may include, for example, sodium amide, potassium amide, lithium amide, magnesium amide, calcium amide, and the like. Suitable Lewis base promoters according to the present invention generally contain nitrogen and may include compounds such as tertiary amines or compounds having nitrogen in a heteroatomic ring. Suitable tertiary amine base promoters can include amines with pendant alkyl or cycloalkyl groups, for example, such as triethylamine, tributylamine, triethylenediamine, N,N-diisopropylethylamine, N,N-diethylcyclohexylamine, and N,N,N′,N′-tetramethylethylenediamine. Suitable compounds having nitrogen in a heteroatomic ring include, for example, pyridine and quinoline, as well as alkylated or tertiary amine-fluctionalized derivatives thereof, for example, such as lutidine and dimethylaminopyridine. If the base promoter includes ammonia, it must also contain another compound capable of facilitating the reaction, such as those listed above. Optionally, but preferably, the base promoter is at least partially soluble in, or compatible with, the melamine, the urea, or the ammonia, especially at the temperature or pressure typically associated with any of the embodiments of the invention. It should be understood that the melamine synthesis processes of the present invention may encompass any reactants known to one of ordinary skill in the art that may reasonably react to form melamine under suitable conditions and that may be facilitated in the presence of a suitable electron-pair donor or basic compound. Advantageously, however, the one or more reactants may include urea, and the temperature and pressure may be readily determined by one of ordinary skill in the art for synthesizing melamine. A slight excess of ammonia may also be present, though not as a reactant, when urea is included as a reactant in the melamine synthesis process according to the invention.

Optionally, but preferably, the purification reaction can be accomplished under high pressure conditions, for example, such as those disclosed for melamine synthesis processes according to the invention. Nevertheless, melamine of any purity that may be purified by any process according to the invention may be synthesized by any known high-pressure or low-pressure process.

The purity of the melamine produced by any of the processes of the present invention is relatively high, such that “high purity” should be understood to be at least about 98%, more preferably at least about 99%, most preferably at least about 99.8%. It should further be understood that “low purity,” as used herein, means between about 96% and 98%, and that “crude,” as used herein, means less than about 96% pure. The melamine purity may be assessed by any suitable method. In one embodiment, the purity of melamine synthesized using a base promoter can be about equivalent to, and preferably higher than, the purity level of crude melamine synthesized by, and/or subsequently purified by, any conventional process for synthesizing or purifying melamine. Alternatively, the melamine synthesized by the process according to the present invention is substantially free of undesired reaction products such that the melamine comprises at least one of: less than about 1200 ppm melam impurities; less than about 200 ppm melem impurities; less than about 200 ppm melon impurities; less than about 400 ppm ureidomelamine impurities; less than about 700 ppm combined ammeline, ammelide, and cyanuric acid impurities; less than about 150 ppm melamine cyanurate impurities; or a combination thereof.

When the base promoter is added during the continuous or discrete-batch syntheses of unpurified melamine, it may be added at any time before the synthesis reaction is substantially complete. The base promoter may be added in any form suitable to facilitate the reaction to form melamine. In a preferred embodiment, the base promoter is dissolved or dispersed in at least one of the reactants as it is being combined with the other reactant(s). For example, the base promoter may be added to molten urea as it is being piped into a reaction zone. In another example, the base promoter may be dissolved in liquid ammonia as it is being piped into a reaction zone.

The phrase “substantially free,” as used herein in reference to a component of a product, means that the product includes not more than about 2%, preferably not more than about 1%, more preferably not more than about 0.2%, most preferably not more than about 0.1% of the component.

The phrase “is not substantially consumed,” as used herein in reference to a promoter during a chemical reaction, means that not more than about 25%, preferably not more than about 10%, more preferably not more than about 5%, most preferably not more than about 1% of the promoter reacts irreversibly with one or more reactants or products during a chemical reaction.

The phrase “crude melamine,” as used herein, should be understood to refer to any product containing melamine and having a melamine purity of less than about 96%.

The term “about,” as used herein in reference to a range of values, should be understood to modify either one or both of the values in the range.

### EXAMPLES

The following examples are only representative of the methods and materials for use in the processes of this invention, and are not to be construed as limiting the scope of the invention in any way.

### Examples 1-2

### Comparison of Melamine Purified With and Without Base Promoter

Low purity melamine made by a high-pressure process with a purity of approximately 96% was used as a starting material for making higher purity melamine. Example 1 shows the result of purification by a process of the prior art, with high-pressure ammonia gas as the only agent used for converting the impurities present into melamine. Example 2 shows the result of a purification process according to the present invention, in which high-pressure ammonia gas and a base promoter were both present to facilitate the conversion of the impurities into melamine. In this case, similar results were obtained when the base promoter was triethylamine as when it was tributylamine. The values reported in Table 1 represent the effects of using triethylamine as the promoter.

TABLE 1

| Reaction Conditions | % Melam Converted | % Melem Converted |
|---|---|---|
| Low Purity Melamine (as | — | — |
| received) | | |
| Example 1: NH₃ only | 99.8+% | 4% |
| Example 2: NH₃ + promoter | 99.8+% | 35% |

In Examples 1 and 2, the temperature of the reactants during each purification process was held at about 398° C. for about 15 minutes, and the ammonia pressure during each purification process was about 950 psig.

As shown in Table 1 above, at least for melem impurities, the process using a combination of high-pressure ammonia gas and the trialkylamine according to the invention was about 9 times as effective at converting the melem impurities from the low purity melamine into melamine as the prior art process using high-pressure ammonia gas alone. There seemed to be no significant difference in percent melam converted between the two Examples. In Example 2, the base promoter was present in about 1% by weight of the low purity melamine.

### Examples 3-6

### Comparison of Melamine Purification Process of the Present Invention With Prior Art Purification Processes

Examples 3 and 4 are parallel experiments to Examples 1 and 2 and were synthesized under conditions intended to approximate those recited in U.S. Pat. No. 5,514,797. Examples 3 and 4 used the same batch of low purity melamine material as that used in Examples 1 and 2. In addition, Example 3, like Example 1 above, shows the result of purification by a process of the prior art, with high-pressure ammonia gas used as the only agent for converting the impurities present into melamine; Example 4, like Example 2 above, shows the result of a purification process of the present invention, in which high-pressure ammonia gas and a base promoter, also present at about 1% by weight, were both used to facilitate the conversion of the impurities into melamine. In Example 4, only tributylamine was used as the base promoter.

In Examples 3 and 4, the temperature of the reactants during each purification process was held at about 398° C. for about 15 minutes, and the ammonia pressure during each purification process was about 930 psig.

Examples 5 and 6 were both prior art purification processes disclosed in U.S. Pat. No. 5,514,797, at ammonia pressures of about 1000 psi and about 1200 psi, respectively, and both at temperatures of about 750° F. (˜398° C.) for about 15 minutes. Both examples 5 and 6 showed results using high-pressure ammonia gas as the only agent used in converting the impurities present into melamine. The low purity melamine used in the purification processes of Examples 5 and 6 was made by a different process than that used in Examples 1-4 and had a purity of approximately 97.5%.

TABLE 2

| | Avg. Press. | Avg. Temp. | | |
|---|---|---|---|---|
| Reaction Conditions | (psig) | (° C.) | % Melem Converted | % Melam Converted |
| Example 3: NH₃ only* | 939 | 398 | 4% | 99.8+% |
| Example 4: NH₃ + promoter* | 964 | 398 | 44% | 99.8+% |
| Example 5: NH₃ only^{#} | 1000 | 398 | −162% | 99.8+% |
| (from U.S. Pat. No. 5,514,797) | | | (Melem FORMED) | |
| Example 6: NH₃ only^{#} | 1200 | 398 | 1% | 99.8+% |
| (from U.S. Pat. No. 5,514,797) | | | | |

*Low purity melamine had 1136 ppm melem impurities and 30300 ppm melam impurities.
^{#}Low purity melamine had 1390 ppm melem impurities and 19215 ppm melam impurities.

As shown in Table 2 above, all the processes examined, both with and without a base promoter, had the similar effect of reducing the concentration of melam impurities. The process using a combination of high-pressure ammonia gas and the tributylamine (Example 4), however, was almost 4 times as effective at converting the melem impurities from the low purity melamine into melamine as the best prior art process using high-pressure ammonia gas alone (Example 3). In the ammonia-only prior art processes of Examples 5 and 6, which were conducted at higher ammonia pressures than those in Examples 3 and 4, little, if any, conversion of melem impurities into melamine was evidenced, and in fact, at the 1000 psi level, melem was created instead of being converted to melamine. The formation of melem in this case (Example 5), without being bound to theory, was thought to have resulted from a further reaction of melam instead of melam breaking down to form melamine.

### Example 7

### Discrete Batch Process for Synthesizing High Purity Melamine Without a Purification Step

Example 7 involves a process for synthesizing high purity melamine in a discrete batch, in which urea is added to a reactor. The urea is subjected to temperature and/or pressure sufficient to melt or liquefy it. Typically, this is accomplished at a temperature of at least about 150° C.±10° C. at atmospheric pressure or greater. The reactor is optionally purged with nitrogen to inhibit or prevent contamination or oxidation. Ammonia gas is subjected to 398° C.±10° C. at a pressure sufficient to liquefy it (e.g., 900 psig to 1000 psig). To this pressurized ammonia, a base promoter is added in an amount of about 1%, based on the total weight of the melamine to be produced in the reactor. The base promoter is dispersed or dissolved in the ammonia. The molten urea is heated to 398° C.±10° C., and then the base promoter and the ammonia are added to the urea at 1400 psig to 1500 psig. The mixture is agitated, and allowed to react for about 50 minutes to 60 minutes, after which the melamine formed by this reaction is cooled and depressurized to facilitate its solidification. Upon retrieval, the melamine has a purity greater than 98%, preferably greater than about 99%, with no purification step.

### Example 8

### Continuous Processes for Synthesizing High Purity Melamine Without a Purification Step

Example 8 involves a continuous process for synthesizing high purity melamine according to the invention. The process of Example 8 involves using a base promoter to help facilitate the reaction of ammonia and urea to form melamine and the conversion of melamine impurities, such as melem, melam, melon, ureidomelamine, and the like, to melamine. In the process of Example 8, the base promoter is introduced in an amount of about 1%, based on the total weight of the melamine produced in the reactor at the time of introduction. Urea is fed into the scrubber at a temperature of about 1 75° C. to 235° C. and at a pressure sufficient to keep the urea molten, and, after being scrubbed with the offgases from the melamine reaction, the molten urea is fed into the melamine synthesis reactor, along with a small amount of ammonia to prevent or inhibit plugging. The base promoter is dispersed or dissolved in the molten urea before the molten urea is fed to the reactor. Highly pure gaseous melamine is separated from the other synthesis offgases in the gas separator and liqeufied, with the other synthesis offgases being sent to the scrubber. Once separated, the liquid melamine is depressurized and rapidly cooled with liquid or gaseous ammonia. Upon retrieval, the solid melamine has a purity greater than 98%, preferably greater than about 99%, with no purification step.

It is to be understood that the invention is not to be limited to the exact configurations and processes described herein. Accordingly, all expedient modifications readily attainable by one of ordinary skill in the art from the disclosure set forth herein, or by routine experimentation therefrom, are deemed to be within the spirit and scope of the invention as defined by the appended claims. In addition, any modifications to the processes herein made for the purposes of converting any laboratory-scale elements of the process to industrial-scale, including modifications associated with converting from discrete-batch to continuous operation, are also deemed to be within the spirit and scope of the invention as defined by the appended claims.

What is claimed is:

1. A process for the production of high purity melamine, which comprises:
heating melamine having a first purity and undesired components therein in a reaction zone to a temperature sufficient to maintain at least a portion of the melamine in a molten state;
adding ammonia to the reaction zone at a temperature and pressure sufficient to decompose undesired components to purify the melamine;
adding a base promoter to the reaction zone in an amount sufficient to facilitate the decomposition of the undesired components in the melamine without significantly decomposing melamine; and
recovering purified melamine having a second purity higher than the first purity.

2. The process of claim 1, wherein the melamine is heated to a temperature from about 345° C. to 450° C.

3. The process of claim 1, wherein the undesired components in the melamine comprise one or more compounds that are decomposable into melamine in the presence of the base promoter.

4. The process of claim 1, wherein the base promoter is present in an amount from about 0.01% to 10%, by weight of the melamine.

5. The process of claim 1, wherein the base promoter is present in an amount from about 0. 1% to 1%, by weight of the melamine.

6. The process of claim 1, wherein the ammonia is added at a pressure from about 200 psi to 2000 psi.

7. The process of claim 1, wherein the recovering comprises sufficiently reducing the pressure of the reaction zone to facilitate retrieval of a high purity melamine solid.

8. The process of claim 1, wherein the base promoter is at least partially soluble in the ammonia or the melamine at the decomposition temperature and pressure.

9. The process of claim 1, wherein the base promoter comprises a nitrogen-containing Lewis base; an amide salt having a general formula [M]^{+x}([NH₂]⁻)ₓ, where M is a monovalent or multivalent metal and where x is from 1 to 6; or a combination thereof.

10. The process of claim 1, wherein the base promoter comprises at least one of sodium amide, potassium amide, lithium amide, magnesium amide, calcium amide, triethylamine, tributylamine, N,N-diisopropylethylamine, N,N-diethylcyclohexylamine, triethylenediamine, N,N,N′,N′-tetramethylethylenediamine, pyridine, quinoline, lutidine, dimethylaminopyridine, or combinations thereof.

11. The process of claim 1, wherein the decomposition reaction between the undesired components in the melamine and the ammonia is sufficient to convert at least about 10% of the undesired components to melamine.

12. The process of claim 1, wherein the decomposition reaction between the undesired components in the melamine and the ammonia is sufficient to convert at least about 50% of the undesired components to melamine.

13. A process for the production of melamine comprising the steps of:
adding a base promoter to one or more reactants for synthesizing melamine to form a mixture, wherein the base promoter is present in an amount sufficient to facilitate a reaction between the one or more reactants to form melamine; and
maintaining the mixture at a temperature and a pressure sufficient to facilitate the reaction between the one or more reactants such that the melamine is formed.

14. The process of claim 13, wherein the one or more reactants is urea or comprises at least one impurity obtainable from a melamine synthesis reaction or purification process.

15. The process of claim 13, wherein the melamine is substantially free of undesired reaction products.

16. The process of claim 13, wherein the base promoter is at least partially soluble in, or compatible with, one of the reactants.

17. The process of claim 13, wherein the base promoter comprises a nitrogen-containing Lewis base; an amide salt having a general formula [M]^{+x}([NH₂]⁻)ₓ, where M is a monovalent or multivalent metal and where x is from 1 to 6; or a combination thereof.

18. The melamine synthesized by the process of claim 13, wherein the melamine comprises a high purity melamine having less than about 20000 ppm of undesired reaction products.

19. The melamine synthesized by the process of claim 13, which is substantially free of undesired reaction products such that the melamine comprises at least one of: less than about 1200 ppm melam impurities; less than about 200 ppm melem impurities; less than about 200 ppm melon impurities; less than about 400 ppm ureidomelamine impurities; less than about 700 ppm combined ammeline, ammelide, and cyanuric acid impurities; less than about 150 ppm melamine cyanurate impurities; or a combination thereof.

20. A crude melamine purification process to remove undesired components, comprising the steps of:
heating crude melamine having undesired components therein in a reaction zone to a temperature sufficient to maintain at least a portion of the crude melamine in a molten state;
adding ammonia to the reaction zone at a temperature and pressure sufficient to decompose undesired components to purify the crude melamine;
adding a base promoter to the reaction zone in an amount sufficient to facilitate the decomposition of the undesired components in the crude melamine without significantly decomposing melamine; and
recovering purified melamine having a second purity greater than the first purity.
